(19) United States
(12) Patent Application Publication
(10) Pub. No.: US 2020/0315688 A1
(43) Pub. Date: Oct. 8, 2020

(54) MONOPOLAR AND BIPOLAR FUNCTIONALITY

(71) Applicant: Ethicon LLC, Guaynabo, PR (US)

(72) Inventors: John Brady, Cincinnati, OH (US); Michael Ehninger, South Jordan, UT (US); Chad P. Boudreaux, Cincinnati, OH (US); Ellen Burkart, Liberty Township, OH (US); Tylor Muhlenkamp, Cincinnati, OH (US); Gregory Bakos, Mason, OH (US); Brian Walter, South Jordan, UT (US); Brian Bertke, Fort Thomas, KY (US); Rafael J. Ruiz Ortiz, Pembroke Pines, FL (US)

(21) Appl. No.: 16/375,454

(22) Filed: Apr. 4, 2019

(51) Int. Cl. A61B 18/14; A61B 18/12

(52) U.S. Cl. CPC A61B 18/1442; A61B 2018/00601; A61B 18/1482; A61B 18/1206

(57) ABSTRACT

In general, surgical devices having monopolar functionality and bipolar functionality are provided. In an exemplary embodiment, a surgical device is configured to selectively apply each of bipolar energy and monopolar energy.

### FIELD

Surgical devices are provided for selectively applying monopolar energy and bipolar energy to tissue.

### BACKGROUND

Various surgical devices can be used for minimally-invasive surgery to compress, transect, and seal different types of tissue. In general, these devices can have an end effector with a pair of opposed jaws that are configured to engage tissue therebetween, and can have a cutting mechanism that is configured to transect tissue engaged by the opposed jaws. The end effector can be configured to apply electrical energy to tissue engaged between the opposed jaws. The application of electrical energy to the engaged tissue can seal and coagulate the tissue, such as to seal tissue being cut by the cutting mechanism to prevent or reduce bleeding.

However, various situations can arise during an operation in which a user wants to apply energy to tissue without having to first grasp tissue between the opposed jaws, such as to selectively apply energy to spots of tissue in a controlled manner without having to clamp and seal an entire section of tissue.

Accordingly, there remains a need for improved energy delivery from surgical devices to tissue.

### SUMMARY

In general, methods and devices are provided herein for selectively applying monopolar energy to tissue adjacent to a surgical instrument and either monopolar or bipolar energy to tissue grasped by the surgical instrument during minimally-invasive surgery.

In one embodiment, a surgical device is provided and includes a housing having an instrument shaft extending therefrom and an end effector assembly having a clamp arm and a conductive member. The instrument shaft can include an inner sleeve and an outer sleeve that is partially disposed around the inner sleeve. The clamp arm can extend distally from the outer sleeve and can be movable between open and closed positions, and the conductive member can extend distally from the inner sleeve. The clamp arm can have a tissue contacting surface and can be configured to translate between a retracted configuration in which it overlaps with and is closed upon a distal portion of the inner sleeve and an extended configuration in which it at least partially overlaps with the conductive member and configured to open and close upon the conductive member.

In some embodiments, the end effector assembly can include a support structure extending distally from the inner sleeve and positioned below and in contact with the conductive member. In other embodiments, the end effector assembly can include at least one slot that extends through at least a portion of at least one of the clamp arm and the conductive member. The slot can be configured to receive a cutting element.

The clamp arm can have a variety of configurations. For example, in some embodiments, an upper portion of the clamp arm cam be pivotally connected to a distal portion of the outer sleeve and the outer sleeve can be movable between retracted and extended configurations. In such embodiments, a lower portion of the clamp arm can include a pin configured to travel within a cam slot formed within a portion of the inner sleeve. When the pin is in at a proximal-most end of the cam slot, the clamp arm can be retracted and closed upon the distal portion of the inner sleeve and distal movement of the pin within the cam slot can move the clamp arm to an open position and advance the clamp arm distally towards the conductive member. In one embodiment, further distal movement of the pin to a distal-most end of the cam slot can move the clamp arm distally into alignment with the conductive member and cause the clamp arm to close upon the conductive member.

In some embodiments, when the clamp arm is in the retracted configuration the device can be configured to treat tissue in a monopolar energy delivery mode with the conductive member. In one embodiment, when the clamp arm is in the extended configuration the device can be configured to treat tissue disposed between the clamp arm and the conductive member. In another embodiment, the tissue contacting surface of the clamp arm can be conductive, and when the clamp arm is in the extended configuration the device can be configured to treat tissue disposed between the clamp arm and the conductive member in a bipolar energy delivery mode.

The conductive member can have variety of configurations. For example, in some embodiments, the conductive member can be a monopolar cutting blade.

In another exemplary embodiment, a surgical device is provided having an instrument shaft that includes an outer sleeve and a clamp arm pivotably coupled to a distal end of the outer sleeve, and a conductive member that extends through the outer sleeve. The instrument shaft can be operably coupled to and extending from a housing. The clamp arm can have a selectively conductive surface formed at least partially thereon. The clamp arm and outer sleeve can be configured to selectively rotate about the conductive member to move cause the device to move between configurations for a monopolar mode of operation and a bipolar mode of operation.

The conductive member can have a variety of configurations. For example, in some embodiments, the conductive member can be substantially L-shaped.

In some embodiments, when in the monopolar mode, the clamp arm can be de-energized and the conductive member can be configured to apply energy to tissue disposed between the clamp arm and the conductive member, and when in the bipolar mode, energy can be delivered between the clamp arm and the conductive member to tissue disposed therebetween. In an exemplary embodiment, when in the monopolar mode, the clamp arm can be de-energized and configured to apply pressure to tissue disposed between the clamp arm and the conductive member.

In other embodiments, when in the monopolar mode, the clamp arm can be de-energized and the selectively conductive surface of the clamp arm can face a first surface of the conductive member, and when in the bipolar mode, the clamp arm can be energized and the selectively conductive surface can face a second surface of the conductive member. In such embodiments, the first surface can have a width that is less than the second surface.

Surgical methods are also provided. In one exemplary embodiment, the method can include positioning at least one of a clamp arm and a conductive member of an end effector assembly of a surgical device in contact with tissue, the clamp arm being coupled to a distal portion of an outer sleeve of the surgical device and the conductive member extending distally from an inner sleeve of the surgical device, actuating an energy source to supply energy to at least one of the clamp arm and the conductive member to treat tissue located adjacent to or in direct contact therewith, and longitudinally translating the clamp arm or the conductive member from a retracted configuration to an extended configuration to position tissue between the clamp arm and the conducive member.

In some embodiments, the clamp arm can be longitudinally translated from its retracted configuration to its extended configuration, and the method can also include moving the clamp arm from an open position to a closed position to grasp tissue positioned between the clamp arm and the conducive member. In other embodiments, when surgical device is in a monopolar energy delivery mode, the step of actuating the energy source can include supplying energy only to the conductive member to treat tissue located adjacent to or in direct contact therewith.

In some embodiments, the clamp arm includes an electrode. In one embodiment, when the surgical device is in a bipolar energy deliver mode, the method can also include actuating the energy source to supply energy to the electrode or the conductive member to treat tissue grasped therebetween. In one embodiment, when the surgical device is in a monopolar energy mode, the step of actuating the energy source can include supplying energy only to the electrode of the clamp arm.

In some embodiments, the method can include longitudinally translating a cutting element from a retracted position to an extended position to cut tissue positioned between the clamp arm and the conductive member.

### BRIEF DESCRIPTION OF DRAWINGS

This invention will be more fully understood from the following detailed description taken in conjunction with the accompanying drawings, in which:

FIG. 1 is a side, partially transparent view of one embodiment of a surgical device that includes inner and outer sleeves and an end effector assembly having a retractable clamping element and a stationary conductive member, showing the outer sleeve and clamping element in a retracted configuration and the clamping element in a first closed position;

FIG. 2A is a magnified view of a distal portion of the surgical device of FIG. 1;

FIG. 2B is a magnified view of a distal portion of the surgical device of FIG. 1, showing the outer sleeve and clamping element in a first extended configuration and the clamping element in an open position;

FIG. 2C is a magnified view of a distal portion of the surgical device of FIG. 1, showing the outer sleeve and clamping element in a second extended configuration and the clamping element in a second closed position;

FIG. 3 is a side, partially transparent view of the surgical device of FIG. 1 operatively coupled to a generator;

FIG. 4 is a perspective view of a compression member of the surgical device of FIG. 1;

FIG. 5A is a magnified perspective partial view of another end effector assembly having a retractable clamping element and a stationary conductive member, each having a slot configured to receive a compression member, showing the clamping element in a retracted configuration;

FIG. 5B is front view of the compression member of FIG. 5A;

FIG. 5C is a side cross sectional view of the end effector assembly of FIG. 5A, showing the clamping element in the retracted configuration and the compression member in a retracted position;

FIG. 5D is a side cross sectional view of the end effector assembly of FIG. 5A, showing the clamping element in an extended configuration and the compression member in an extended position;

FIG. 6A is a magnified view of another end effector assembly having a support structure;

FIG. 6B is a cross-sectional view of the end effector assembly taken at line 6B-6B.

FIG. 7A is a side view of another embodiment of a surgical device that includes an outer sleeve and an end effector assembly having a upper jaw and a retractable lower jaw, showing the lower jaw in an extended configuration;

FIG. 7B is side view of the surgical device of FIG.7A, showing the lower jaw in a retracted configuration;

FIG. 8 is an isometric view of a distal portion of the surgical device of FIG. 7A;

FIG. 9 is an isometric view of a distal portion of another embodiment of a surgical device that includes an outer sleeve and an end effector assembly having a clamping element and a ultrasonic blade;

FIG. 10 is a side, partially transparent view of another embodiment of a surgical device that includes an instrument shaft and an end effector assembly having a rotatable clamping element and a stationary conductive member, showing the clamping element in an open position;

FIG. 11A is a side view of the conductive member of FIG. 10;

FIG. 11B is a front view of the conductive member of FIG. 10;

FIG. 12A is a side, partially transparent view of the instrument shaft and the end effector assembly of FIG. 10, showing the clamping element in a zero rotation position and in a first closed position;

FIG. 12B is a face view of the proximal end of the instrument shaft of FIG. 12A;

FIG. 13A is a side, partially transparent view of the instrument shaft and the end effector assembly of FIG. 10, showing the clamping element in a first rotation position and in a second closed position;

FIG. 13B is a face view of the proximal end of the instrument shaft of FIG. 13A;

FIG. 14A is a side, partially transparent view of the instrument shaft and the end effector assembly of FIG. 10, showing the clamping element in a second rotation position and in a third closed position;

FIG. 14B is a face view of the proximal end of the instrument shaft of FIG. 14A;

FIG. 15A is an opposing side, partially transparent view of the instrument shaft and the end effector assembly of FIG. 10, showing the clamping element in a third rotation position and in a fourth closed position;

FIG. 15B is a face view of the proximal end of the instrument shaft of FIG. 15A;

FIG. 16 is a schematic view of a portion of the surgical device of FIG. 9 operatively coupled with one embodiment of a generator;

FIG. 17 is a side, partially transparent view of another embodiment of a surgical device that includes an instrument shaft and an end effector assembly having a rotatable clamping element and a stationary conductive member, showing the clamping element in an open position and zero rotation position;

FIG. 18A is a side, partially transparent view of a proximal end of the instrument shaft of FIG. 17 coupled to a shroud that is operably coupled to one embodiment of a generator;

FIG. 18B is a top, partially transparent view of a proximal end of the instrument shaft of FIG. 17 coupled to a shroud that is operably coupled to one embodiment of a generator, in which the clamping element is in a first rotation position;

FIG. 18C is a side, partially transparent view of a proximal end of the instrument shaft o of FIG. 17 coupled to a shroud that is operably coupled to one embodiment of a generator, in which the clamping element is in a second rotation position; and

FIG. 18D is a top, partially transparent view of a proximal end of the instrument shaft of FIG. 17 coupled to a shroud that is operably coupled to one embodiment of a generator, in which the clamping element is in a third rotation position.

### DETAILED DESCRIPTION

Certain exemplary embodiments will now be described to provide an overall understanding of the principles of the structure, function, manufacture, and use of the devices, systems, and methods disclosed herein. One or more examples of these embodiments are illustrated in the accompanying drawings. Those skilled in the art will understand that the devices, systems, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. A person skilled in the art will appreciate that a dimension may not be a precise value but nevertheless be considered to be at about that value due to any number of factors such as manufacturing tolerances and sensitivity of measurement equipment. Sizes and shapes of the systems and devices, and the components thereof, can depend at least on the anatomy of the subject in which the systems and devices will be used, the size and shape of components with which the systems and devices will be used, and the methods and procedures in which the systems and devices will be used.

Various exemplary methods, devices, and systems are provided for applying monopolar energy and/or bipolar energy to tissue from a surgical instrument, such as a minimally-invasive surgical instrument with an end effector assembly having a clamping element or clamp arm and a conductive member for treating tissue (e.g., transecting and/or sealing of tissue in close contact with, adjacent to, or in direct contact with the conductive member and/or of tissue grasped between the clamping element and the conductive member). While spot coagulation, non-clamping sealing and/or hemostasis, marking tissue, cutting or searing tissue, etc., can be accomplished by applying energy to tissue in a monopolar mode of operation via the conductive member, it can be beneficial to incorporate a clamping element that can apply pressure to the tissue grasped between the clamping element and the conductive member to help facilitate sealing of large tissue structures (e.g., vessels). Alternatively, the conductive member can be replaced with a non-conductive member and the clamping element can be configured to apply energy to tissue.

In some instances, the clamping element can be designed as a return electrode such that energy can be applied in a bipolar mode between the conductive member and the clamping element, and thus treat (e.g., seal) tissue grasped therebetween (e.g., vessels). Alternatively, the conductive member can be designed as the return electrode in which the clamping element can be configured to apply energy to tissue. In either instance where the clamping element or the conductive member is the return electrode, the bipolar mode facilitates a more precise and controlled energy path since the electrosurgical current in the patient is restricted to the grasped tissue between the clamping element and the conductive member. As such, the device can be switchable between a monopolar energy delivery mode and a bipolar energy delivery mode.

Further, in some surgical situations in can be useful or necessary to move the end effector assembly in different orientations to access a surgical site. While an end effector assembly with a clamping arm can rotate in its entirety, it can be beneficial to have the clamping arm rotate independent of the conductive member. For example, in instances where the conductive member has a substantially L-shaped configuration, rotation of the clamping element about the conductive member can facilitating multiple geometry states of the end effector assembly. Additionally, the rotation of the clamping arm about the conductive member can allow the device to be switchable between a monopolar mode and a bipolar mode.

In one embodiment, a surgical device is provided having a housing, which can be in the form of a handle, with an instrument shaft extending distally therefrom. An end effector assembly can be operatively connected to a distal end of the instrument shaft, and the end effector assembly can have a clamping element that is movable between open and closed positions and a conductive member that is configured to conduct energy through tissue adjacent to or in direct contact therewith. As used herein, “clamping element” is used synonymously with “clamp arm” and “clamping arm.” The clamping element can translate between a retracted configuration in which it is aligned with and closed upon a portion of the instrument shaft and an extended configuration in which it is aligned with the conductive member and configured to open and close upon the conductive member. When the clamping element is in the retracted configuration, the conductive member can be fully exposed to treat tissue in a monopolar mode, and when the clamping element is in the extended configuration, the clamping element and the conductive member can cooperate to grasp and treat tissue therebetween in the monopolar mode. Further, in certain embodiments, the clamping element can have a conductive tissue-contacting surface such that, when the clamping element is in the extended configuration, the clamping element and the conductive member can cooperate to grasp and treat tissue therebetween in a bipolar mode.

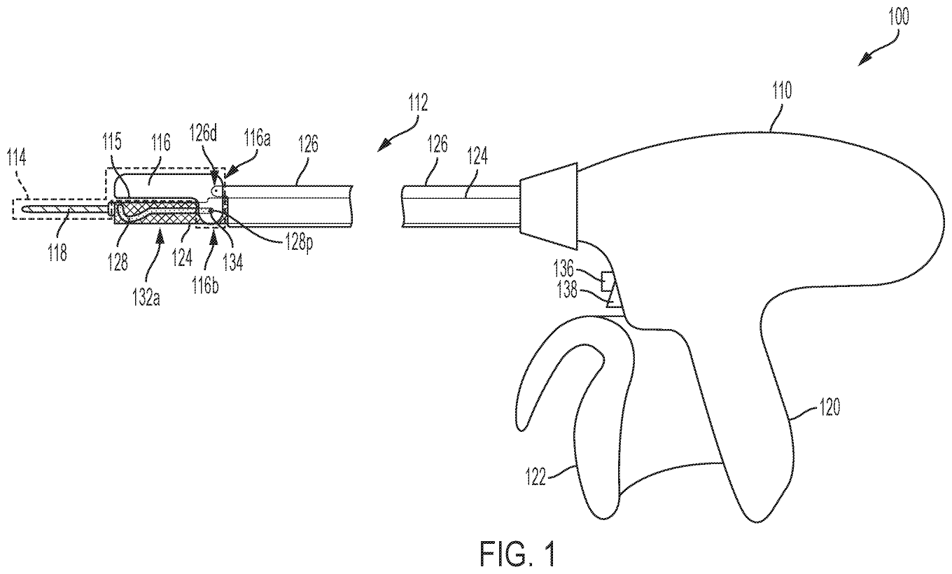

FIG. 1 illustrates an embodiment of a surgical device 100 having an end effector assembly 114 having a retractable clamping element 116 and a stationary conductive member 118 that is configured to cut and seal tissue. For purposes of simplicity, certain components of the surgical device 100 are not illustrated in FIG. 1. As described in more detail below, the surgical device 100 is configured to treat tissue in a monopolar energy delivery mode with the stationary conductive member.

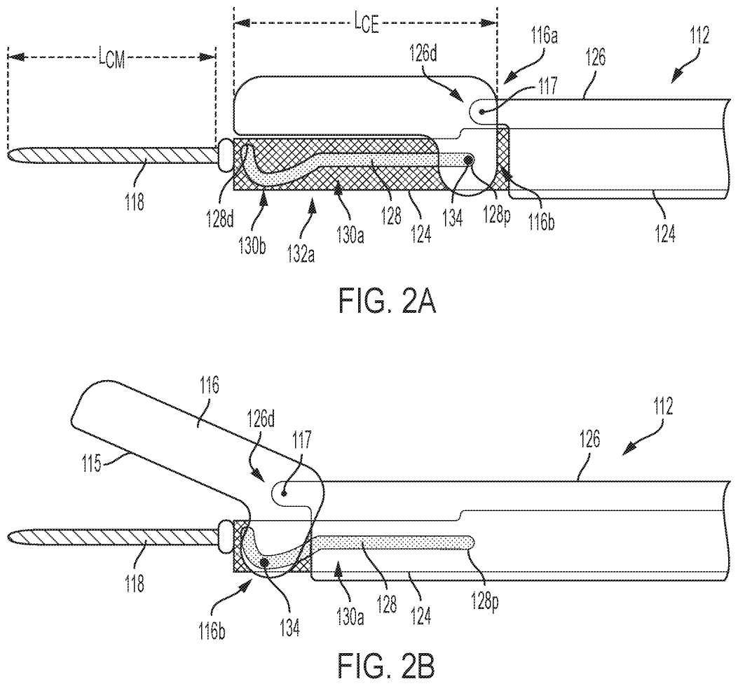

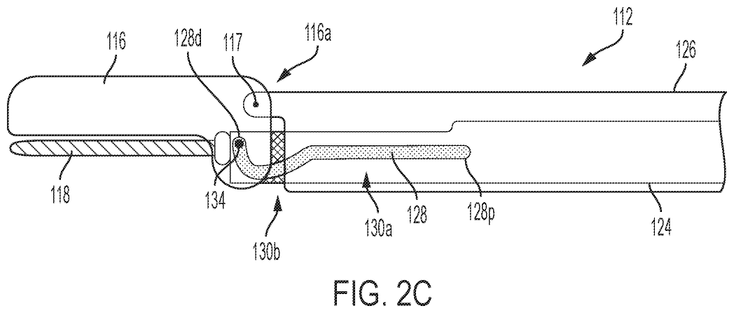

The illustrated surgical device 100 includes a housing 110, an instrument shaft 112, and the end effector assembly 114. The housing 110 can be any type of pistol-grip, scissor grip, pencil-grip, or other type of housing known in the art that is configured to carry various actuators, such as actuator levers, knobs, triggers, sliders, etc. for actuating various functions such as rotating, articulating, approximating, and/or firing a component of the end effector assembly 114. In the illustrated embodiment, the housing 110 is coupled to a stationary grip handle 120 and a closure grip handle 122 is configured to move relative to the stationary grip handle 120 to open and close the clamping element 116. The movement of the closure grip handle 122 is also configured to cause the clamping element 116 to translate relative to the stationary conductive member 18 between a retracted configuration, as shown in FIGS. 1 and 2A, and at least first and second extended configurations, as shown in FIGS. 2B and 2C, respectively.

As shown, the shaft 112 extends distally from the housing 110 and includes an inner sleeve 124 and outer sleeve 126 that is partially disposed around the inner sleeve 124. The inner and outer sleeves 124, 126 each have an elongate tubular configuration and define a lumen extending therethrough for carrying mechanisms for actuating the end effector assembly 114. The outer sleeve 126 is operably coupled to the closure grip handle 122, and therefore movable relative to the inner sleeve 124 between retracted and extended configurations.

The conductive member 118 is coupled to and distally extends from the inner sleeve 124. In other embodiments, the conductive member 118 can be coupled to and distally extend from the outer sleeve 126. The conductive member 118 is configured to receive and apply energy to tissue (e.g., tissue adjacent to or in direct contact therewith). The conductive member 118 can have a variety of shapes and sizes and can be made from a variety of electrically-conductive materials, such as metal. In this illustrated embodiment, the conductive member 118 has a substantially elongate and linear shape.

The inner sleeve 124 includes a cam slot 128 that is formed within a portion thereof, as shown in FIGS. 1-2C. The cam slot 128 can have a variety of configurations. In this illustrated embodiment, the cam slot 128 has a substantially linear elongated portion 130a, which transitions to a substantially c-shaped portion 130b at a distal end thereof. In this illustrated embodiment, the cam slot 128 extends partially through the inner sleeve 124 thereby forming a channel, but a person skilled in the art will appreciate that the cam slot 128 can extend entirely through the inner sleeve 124. The size and shape of the cam slot 128 can vary. For example, as shown, the cam slot 128 partially extends along a length of a distal portion 132a of the inner sleeve 124. A person skilled in the art will appreciate that the size and shape of the cam slot 128 can be based at least in part on the size and shape of the clamping element 116 and conductive member 118. As discussed in more detail below, the cam slot 128 is configured to receive and house a pin 134 that is coupled to the clamping element 116 and configured to selectively slide within the cam slot 128 when a force is applied to the outer sleeve 126, which is operatively coupled to the pin 134. Such sliding movement of the pin 134 within the cam slot 128 causes the outer sleeve 126 to translate relative to the inner sleeve, and consequently, the clamping element 116 to translate and open/close relative to the stationary conductive member 118.

The clamping element 116 can have a variety of sizes, shapes, and configurations. As shown in FIG. 1, the clamping element 116 is a jaw having a tissue contacting surface 115. The clamping element 116 extends distally from the outer sleeve 126. In other embodiments, the clamping element can extend distally from the inner sleeve 124. The clamping element 116 is moveable between closed and open positions, as discussed in more detail below. While the illustrated clamping element 116 has a substantially elongated shape that is curved along a longitudinal length (L_{CE}) thereof, a person skilled in the art will appreciate that the clamping element 116 can have a straight shape or curve in various other directions. The clamping element 116 can have any suitable axial length for engaging tissue disposed between the clamping element 116 and the stationary conductive member 118. As such, the length of the clamping element 116 can be selected based at least upon the axial length (L_{CM}) of the stationary conductive member 118 and the targeted anatomical structure for transection and/or sealing.

As shown, an upper portion 116a of the clamping element 116 is pivotally connected to a distal portion 126d of the outer sleeve 126 via a pivot pin 117. Further, a lower portion 116b of the clamping element 116 includes pin 134 that is configured to travel within the cam slot 128 formed within the distal portion 132a of the inner sleeve 124. As a result, the clamping element 116 is configured to translate from a retracted configuration in which the clamping element 116 is in a first closed position and the pin 134 is in a proximal-most end 128p of the cam slot 128, as shown in FIGS. 1 and 2A, to a first extended configuration, as shown in FIG. 2B, in which the pin 134 moves in a distal direction through the linear elongated portion 130a and into the c-shaped portion 130b of the cam slot 128 causing the clamping element 116 to move into an open position, and to a second extended configuration, as shown in FIG. 2C, in which the pin 134 moves to the distal-most end 128d of the cam slot 128 causing the clamping element 116 to move into a second closed position. In some embodiments, the lower portion 116b of the clamping element 116 can include an additional pin that is configured to travel within an additional cam slot formed within the distal portion 132a of the inner sleeve 124. The additional pin and pin 134 can be configured to concurrently travel along their respective cam slots. Further, the additional pin and pin 134 can have similar structural configurations and the additional cam slot and cam slot 128 can have similar structural configurations.

When in the retracted configuration, the clamping element 116 overlaps with and is closed upon the distal portion 132a of the inner sleeve 124. This allows the conductive member 118 to be fully exposed and apply energy, in a monopolar mode of operation, to tissue adjacent to or in contact with any surface thereof, as discussed in more detail below. When the clamping element 116 is translated to its first extended configuration, the clamping element 116 moves into the open position and partially overlaps with the stationary conductive member 118. This allows tissue to be positioned between the clamping element 116 and the stationary conductive member 118. And, when the clamping element 116 is translated to the second extended configuration, the clamping element 116 moves into the second closed position. Thus, when the clamping element 116 is in the second closed position, a longitudinal axis of the clamping element 116 can be substantially parallel to a longitudinal axis of the conductive member 118, and as a result, the clamping element 116 and conductive member 118 can act to engage or grasp tissue therebetween. That is, while the conductive member 118 is applying energy to the grasped tissue, the clamping element 116 can apply pressure to the grasped tissue, thereby enhancing the sealing capabilities of the conductive member 118 during the monopolar mode of operation.

In the illustrated embodiment, the closure grip handle 122 is operably coupled to the outer sleeve 126 and is configured to pivot relative to and toward and away from stationary grip handle 120. As such, movement of the closure grip handle 122 causes the outer sleeve 126 to translate between its retracted and extended configurations, and thus the clamping element 116 to translate between its retracted and extended configurations and its closed and open positions. In particular, the closure grip handle 122 can be movable between a first or initial position, a second or intermediate position, and a third or final position. In the first position, the closure grip handle 122 can be angularly offset and spaced apart from the stationary grip handle 120 at a first distance, and the outer sleeve 126 and clamping element 116 are in their respective retracted configurations. In at least some embodiments the closure grip handle 122 is biased to the first position with the clamping element 116 being in its retracted configuration and first closed position, as shown in FIG. 1. In the second position, the closure grip handle 122 can be angularly offset and spaced apart from the stationary grip handle 120 at a second distance that is less than the first distance, and the outer sleeve 126 and clamping element 116 are in their first extended configurations. In the third position, the closure grip handle 122 is positioned adjacent to, or substantially in contact with, the stationary grip handle 120, and the outer sleeve 126 and clamping element 116 are in their second extended configurations. Further description of embodiments of end effector assembly opening and closing is provided in U.S. Pat. No. 10,010,309 entitled “Surgical Device With Overload Mechanism” filed Oct. 10, 2014, which is hereby incorporated by reference in its entirety.

The closure grip handle 122 can use manual or powered components. In manual embodiments the closure grip handle 122 is configured to be manually moved (e.g., by a user directly or by a user indirectly via robotic surgical control) to manually translate the outer sleeve 126 and clamping element 116 and the clamping element 116 to close upon the stationary conductive member 118 using various components, e.g., gear(s), rack(s), drive screw(s), drive nut(s), etc. disposed within the housing 110 and/or instrument shaft 112.

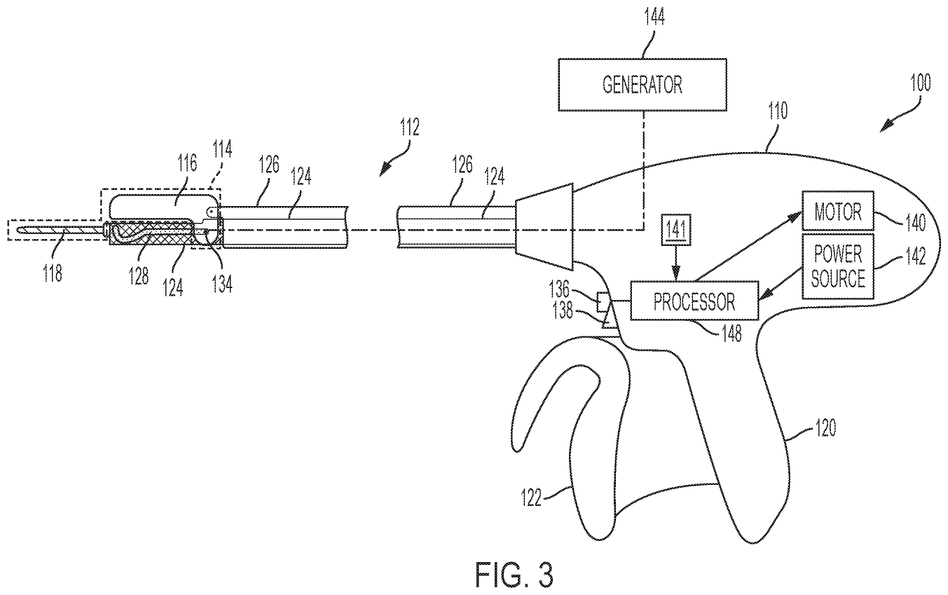

In powered embodiments, the closure grip handle 122 is configured to be manually moved (e.g., by a user directly or by a user indirectly via robotic surgical control), thereby causing the outer sleeve 126 and clamping element 116 to distally translate and the clamping element 116 to close upon the stationary conductive member 118 either fully electronically or electronically in addition to manual power. In this illustrated embodiment, as shown in FIG. 3, the device 100 is powered and includes a motor 140, a power source 142, and a processor 148, which in this illustrated embodiment are each disposed in the housing 110. Manual movement of the closure grip handle 122 is configured to cause the processor 148 to transmit a control signal to be sent to the motor 140, which is configured to interact with various components of the device 100 to cause the outer sleeve 126 and clamping element 116 to distally translate and the clamping element 116 to close upon the stationary conductive member 118. The power source 142 is configured to provide on-board power to the processor 148 and the motor 140. In other embodiments, the processor 148 and/or the motor 140 can be configured to be powered instead, or additionally, with an external power source. The device 100 can include one or more sensors 141 to facilitate powered end effector opening and closing and/or other device features, such as tissue cutting. Various embodiments of such sensors are further described in U.S. Pat. No. 7,416,101 entitled “Motor-Driven Surgical Cutting And Fastening Instrument With Loading Force Feedback” filed Jan. 31, 2006 and U.S. Pat. No. 9,675,405 entitled “Methods And Devices For Controlling Motorized Surgical Devices” filed Apr. 8, 2014, which are hereby incorporated by reference in their entireties. Further description of embodiments of end effector opening and closing is provided in U.S. Pat. No. 10,010,309 entitled “Surgical Device With Overload Mechanism” filed Oct. 10, 2014, which is hereby incorporated by reference in its entirety. A person skilled in the art will appreciate that in powered embodiments, including for robotic systems, there need not be a closure grip handle 122 and instead an actuator can effect movement of the outer sleeve 126.

In at least some embodiments, the closure grip handle 122 can also interact with one or more locking features to lock the closure grip handle 122 relative to the stationary grip handle 120, as will be appreciated by a person skilled in the art. For example, the locking feature can automatically engage when the closure grip handle 122 substantially contacts the stationary grip handle 120 or the locking feature can automatically engage at each position the closure grip handle 122 is pivoted through, such as via ratcheting.

The surgical device 100 can also have one or more additional actuators that can be separate from the closure grip handle 122, such as a sealing actuator 136 to apply energy to tissue. While the actuator 136 can have various configurations, the illustrated actuator 136 is a button or trigger that can be depressed by a user and can activate various elements in the device 100 to cause energy to be delivered to the end effector assembly. As shown in FIG. 3, the housing 110 of the surgical device 100 can include other components for operating the device, such as a motor 140, a power source 142, a generator 144, and/or a processor 148, as well as one or more sensors 141.

The device 100 can also include various components for delivering energy, such as radiofrequency (RF) or ultrasound energy, to tissue, and these components can be disposed at various locations in the device 100, such as in the housing 110 and/or in the conductive member 118 or in both the conductive member 118 and the clamping element 116. The sealing actuator 136 can be coupled to the processor 148, and the processor 148 can be coupled to the motor 140, the power source 142, and/or the generator 44 (as well as any sensors provided). Depressing the sealing actuator 136 can send a signal to the processor 148, which can cause delivery of energy from the generator 144 and/or the power source 142 to the conductive member 118. The energy produced by the generator 144 can be used to cut and/or coagulate tissue. In various embodiments, energy actuation can allow selective application of more than one electrical waveform to the conductive member 118, such as having one continuous low-voltage waveform for tissue cutting and another interrupted high-voltage waveform for tissue and blood coagulation.

The generator 144 can be incorporated into the housing 110 or can be a separate unit, as shown in FIG. 3, that is electrically connected to the surgical device 100. The generator 144 can be any suitable generator known in the art, such as an RF generator or an ultrasound generator. The lumen of the inner sleeve 124 and/or outer sleeve 126 can carry electrical leads, conductive members, wires, etc. that can deliver electrical energy to components of the end effector assembly 114, e.g., the conductive member 118, upon depression of the sealing actuator 136. Both the generator 144 and the power source 142 can be battery-powered, can include batteries therein, and/or can be coupled to an external power source, such as an electrical outlet. Further description of embodiments of energy application by surgical devices is provided in U.S. Pat. No. 10,010,366 entitled “Surgical Devices And Methods For Tissue Cutting And Sealing” filed Dec. 17, 2014, U.S. Pat. No. 7,169,145 entitled “Tuned Return Electrode With Matching Inductor” filed Nov. 21, 2003, U.S. Pat. No. 7,112,201 entitled “Electrosurgical Instrument And Method Of Use” filed Jan. 22, 2003, and U.S. Patent Pub. No. 2017/0135712 entitled “Methods And Devices For Auto Return Of Articulated End Effectors” filed Nov. 17, 2015, which are hereby incorporated by reference in their entireties.

In the illustrated embodiment, the conductive member 118 is a monopolar cutting blade that is in electrical communication with the generator 144. A patient return electrode, not shown, which can be in the form of a ground pad, is also coupled to the generator 144, and, before energizing the conductive member 118, the patient return electrode is placed on the patient's body. When activated, the generator 144 creates electrosurgical energy, such as radio frequency (RF) electrical energy, that flows through the conductive member 118, thereby energizing the conductive member 118 and then from the conductive member 118 into the tissue. The energy then passes through the patient as it completes the circuit from the conductive member 118 to the patient return electrode and then returns to the generator 144 via a connector (not shown). As such, the generator 144 can regulate the electrical energy delivered to the conductive member 118, and effectively to the patient, during surgery. In various embodiments, energy actuation can allow selective application of more than one electrical waveform to the conductive member 118, as discussed above.

In use, while the clamping element 116 is in its retracted configuration, the surgical device 100 is configured to treat tissue in a monopolar energy delivery mode with the conductive member 118. That is, the conductive member 18 can be manipulated within a patient's body and the sealing actuator 136 can be actuated to energize the conductive member 118 via the generator 144. As a result, the conductive member 18 can delivery energy to, and thus treat, tissue that is adjacent to or in direct contact with any surface of the conductive member 118. During use in the monopolar energy delivery mode, the closure grip handle 122 can be moved from its first position to its second position, to cause the outer sleeve, and thus the clamping element 116, to distally translate to a first extended configuration. Once in the first extended configuration, the surgical device 100 can be manipulated to engage tissue (e.g., a vessel) between the opened clamping element 116 and the conductive member 118. Further, the closure grip handle 122 can be moved from its second position to its third position to cause the outer sleeve, and thus the clamping element 116, to distally translate to a second extended configuration. As a result, the clamping element closes upon the conductive member 118 and compresses the engaged tissue against the conductive member 118 to help effect sealing of the engage tissue by the conductive member 118.

While energy can be delivered to tissue grasped between the clamping element 116 and the conductive member 118 via the conductive member 118 when the device is in a monopolar energy delivery mode, in some embodiments, the device 100 can be further configured with a bipolar energy delivery mode. For example, the clamping element 116 can include a return electrode that can be at least partially disposed on a surface of the clamping element 116, e.g., the tissue-contacting surface 115, or alternatively, partially disposed within the clamping element 116 and defining at least a portion of the tissue-contacting surface 115. In such instances, the return electrode is electrically isolated from the conductive member 118 such that energy can be applied to grasped tissue from the conductive member 118 and can have a return path through the clamping element 116. As a result, a surgeon need not change devices to switch between monopolar and bipolar modes of operation. Instead, the same device can be conveniently configured for use in a bipolar energy delivery mode and a monopolar energy delivery mode any number of times as desired by a surgeon or other medical professional. Additionally, a hospital or other buyer of the surgical device need only purchase a single device, instead of two devices, in order to provide its medical professionals with the ability to apply energy in bipolar and monopolar delivery modes, thus reducing overall costs and/or helping to reduce operating room clutter.

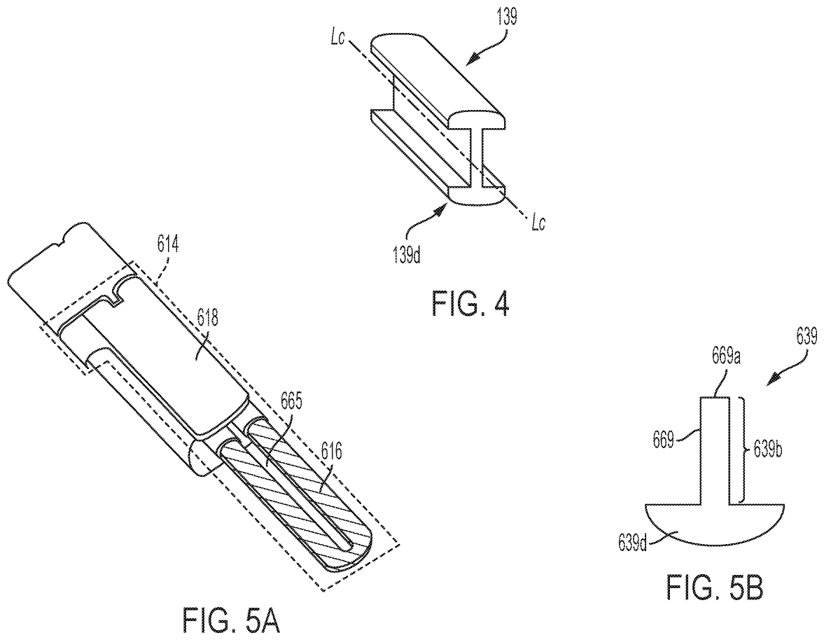

The surgical device 100 can also have a cutting actuator 138 to advance a cutting assembly. While the actuator 138 can have various configurations, the illustrated actuator 138 is a button or trigger that can be depressed by a user and can activate various elements in the device 100 to advance a component of the cutting assembly 114. For example, the cutting actuator 136 can be in mechanical or electrical communication with various gear(s), rack(s), drive screw(s), drive nut(s), motor(s), and/or processor(s). The cutting assembly can be configured to transect tissue captured between the clamping element 116 and the conductive member 118, and it can be sized and shaped to transect or cut various thicknesses and types of tissue. In one exemplary embodiment, as shown in FIG. 4, the cutting assembly can include an I-beam compression member 139 that travels along a longitudinal axis (L_{C}) through a slot formed in at least the conductive member 118 to transect tissue using a cutting element on the distal end 139d of the compression member 139. Alternatively, the I-beam compression member can travel along the longitudinal axis (L_{C}) through slots formed in the clamping element 116 and the conductive member 118 to pull the them into a parallel orientation, to compress tissue therebetween, and to transect tissue using a cutting element on the distal end 139d thereof.

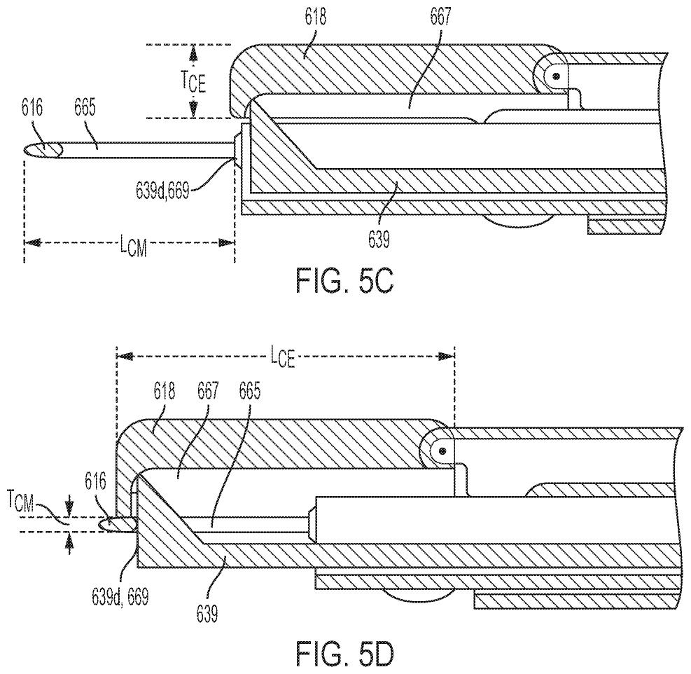

FIGS. 5A and 5C-5D illustrate an exemplary end effector assembly 614 having a first slot 665 formed within the conductive member 616 and a second slot 667 formed in the clamping element 618. Each slot 665, 667 is configured to receive a T-shaped compression member 639, that includes a cutting element or edge 669. In this illustrated embodiment, and as shown in more detail in FIG. 5B, the cutting element or edge 669 is formed on the vertical segment 639b of the distal end 639d of the T-shaped compression member 639. Each slot 665, 667 can have a variety configurations. As shown, the first slot 665 extends along a portion of the axial length (L_{CM}) of the conductive member 618 and completely through the thickness (T_{CM}) of the 618 conductive member. The second slot 667 extends along a portion of the axial length (L_{CE}) of the clamping element 618. In use, upon actuation of a cutting actuator, such as cutting actuator 138 shown in FIGS. 1-3, the compressing member 639, and thus the cutting element 669, can move through the slots 665, 667 (e.g., from a retracted position (FIG. 5C) to an extended position (FIG. 5D) to transect tissue positioned between the conductive member 616 and the clamping element 618. Further, as shown in FIGS. 5C-5D, the second slot 667 extends through only a portion of the thickness (T_{CE}) of the clamping element 618. In this way, during use, inadvertent cutting of adjacent tissue (e.g., by the tip 669a of the cutting element 669) that is not positioned between the conductive member 616 and the clamping element 618 can be avoided. A person skilled in the art will appreciate that in other embodiments, the second slot 667 can extend completely through the thickness of the clamping element 618. Further, a person skilled in the art will appreciate that the compression member 639 can have other suitable shapes and sizes.

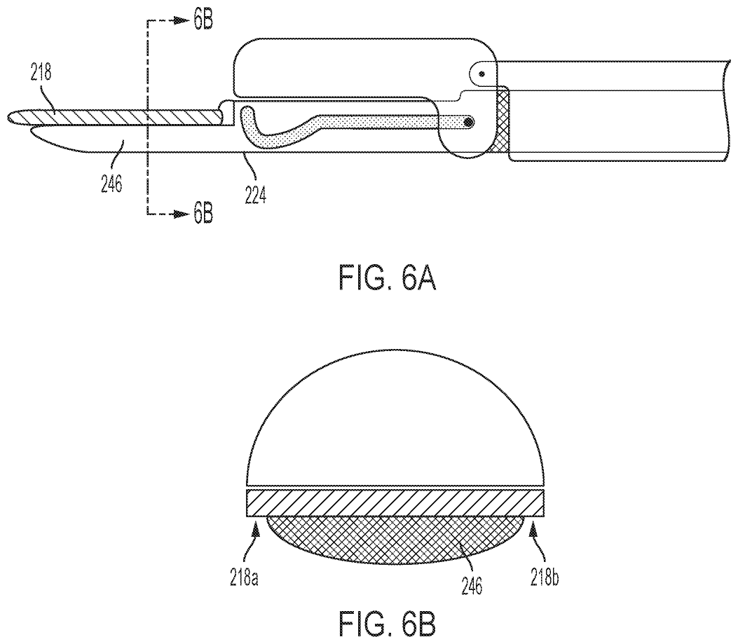

In some embodiments, the end effector assembly can include a support structure that prevents the conductive member from bending while in contact with and compressing tissue. The support structure can have a variety of configurations. For example, as shown in FIGS. 6A-6B, the support structure 246 can extend distally from the inner sleeve 224 and positioned below and in contact with the conductive member 218. While the support structure 246 can have variety of shapes and sizes, as shown in FIG. 6B, the support structure 246 typically has a width that is less than the width of the conductive member 218 such that the edges 218a, 218b of the conductive member 218 can remain exposed for treating tissue. The support structure 246 can be formed as an extension of the inner sleeve 224, or as a separate structure that is subsequently coupled to and extends distally from the inner sleeve 224. The support structure 246 can be formed of a variety of materials, such as rigid non-conductive materials. In one embodiment, the support structure 246 can be formed of the same material as the inner sleeve 224. Further, in some embodiments, the support structure 246 can include a slot that extends along at least a portion of the length of the support structure 246 and configured for receiving a compression member, like compression member 639 in FIGS. 5B-5D.

In some embodiments, the end effector assembly can include an upper jaw and a retractable lower jaw in which the upper jaw is movable between open and closed positions and configured to conduct energy through tissue adjacent to or in direct contact therewith. When the lower jaw is in the retracted configuration, the upper jaw can be fully exposed to treat tissue in a monopolar mode of operation, and when the lower jaw is in the extended configuration, the upper and lower jaws can cooperate to grasp and treat tissue therebetween for treatment in the monopolar mode of operation. Further, the upper jaw can have a hook tip that can be used for dissecting tissue prior to, during, or subsequent to the application of energy to tissue.

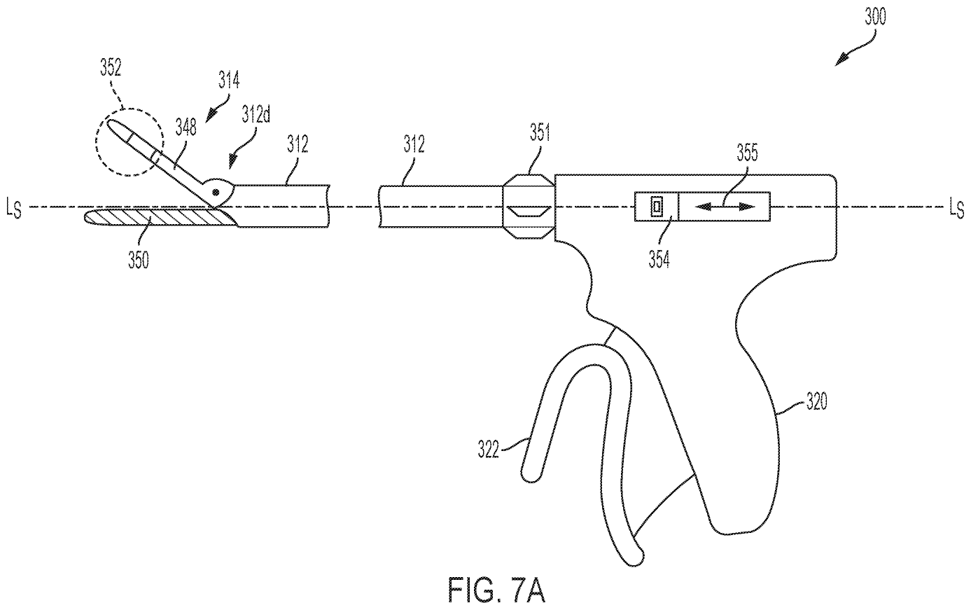

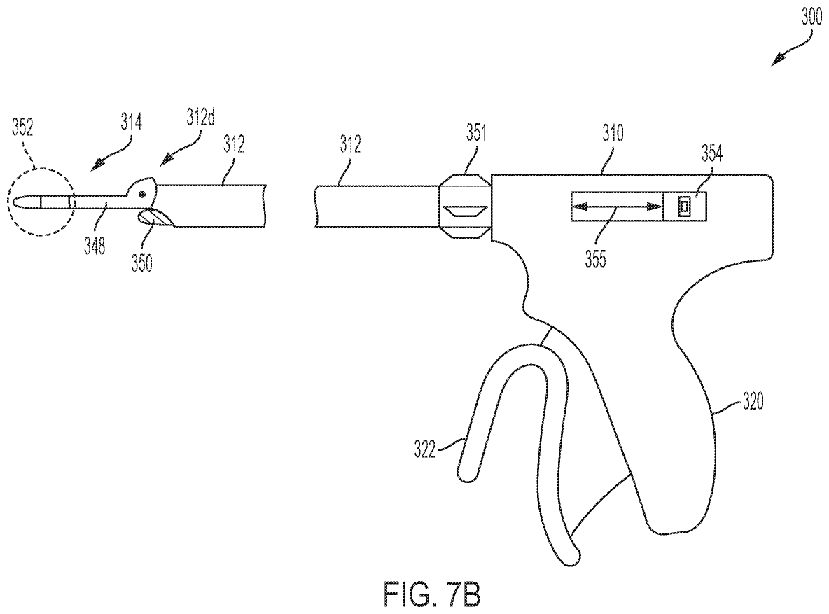

FIGS. 7A and 7B illustrate an embodiment of a surgical device 300 having an end effector assembly 314 that includes an upper jaw 348 and a retractable lower jaw 350. Aside from the differences described in detail below, the surgical device 300 can be similar to the surgical device 100 shown in FIG. 3 and therefore common elements are not further described in detail herein. Further, for purposes of simplicity, certain components of the surgical device 300 are not illustrated in FIGS. 7A and 7B.

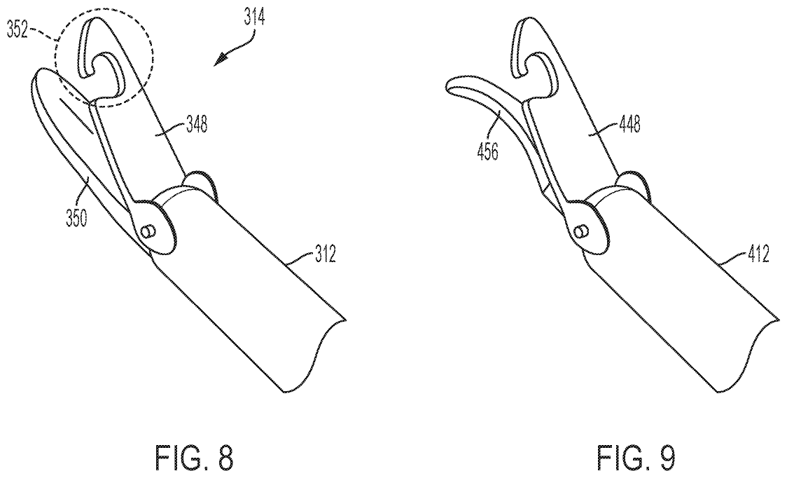

In this illustrated embodiment, the upper jaw 348 is pivotally connected to and distally extending from a distal portion of 312d of the instrument shaft 312. The upper jaw is configured to pivot relative to the instrument shaft 312 and relative to the lower jaw 350. While the upper jaw 348 can have a variety of configurations, the upper jaw 348 can be configured to receive and apply energy to tissue that is adjacent to or in direct contact therewith. As such, the upper jaw 348 can function similarly to conductive member 118 shown in FIGS. 1-3 for purposes of applying energy to tissue to effect cutting and/or sealing thereof. The upper jaw 348 can therefore be formed of a variety of electrically-conductive materials, such as metal. As shown in FIG. 8, the upper jaw 348 can include a hook tip 352 on the distal end thereof that can be configured to dissect tissue. The hook tip 352 can have a variety of shapes and sizes. For example, as shown in FIG. 8, the hook tip 352 is substantially c-shaped. The shape and size of the hook tip 352 can be based at least upon the targeted anatomical structure for transection.

Further, in the illustrated embodiment actuation of the closure grip handle 322, e.g., movement towards the stationary grip 320, can cause the upper jaw 348 to articulate and/or pivot towards and away from the longitudinal axis (L_{S}) of the instrument shaft 312. The closure grip handle 322, like closure grip handle 122, can use manual or powered components. In manual embodiments the closure grip handle 322 is configured to be manually moved (e.g., by a user directly or by a user indirectly via robotic surgical control) to manually articulate and/or pivot the upper jaw 348 using various components, e.g., gear(s), rack(s), drive screw(s), drive nut(s), etc. disposed within the housing 310 and/or instrument shaft 312. In powered embodiments, the closure grip handle 322 (or another actuator) is configured to be manually moved (e.g., by a user directly or by a user indirectly via robotic surgical control), thereby causing articulation and/or pivoting of the upper jaw either fully electronically or electronically in addition to manual power. In this illustrated embodiment, the device 300 is powered similar to surgical device 100 and therefore not described in detail herein. The upper jaw 348 can also be rotated about the longitudinal axis of the instrument shaft 312 by rotating a knob 351 that is coupled to the housing 310.

As further shown in FIG. 7A, the lower jaw 350 extends distally from the distal end 312d of the instrument shaft 312. Unlike the upper jaw 348, the lower jaw 350 is configured to translate relative to the instrument shaft 312, and thus relative to the upper jaw 348. As a result, the lower jaw 350 can move from an extended configuration, as shown in FIG. 7A, to a retracted configuration, as shown in FIG. 7B, and vice versa. When in the retracted configuration, the lower jaw 350 is received within the instrument shaft 312, allowing the upper jaw 348 to be fully exposed and apply energy to tissue adjacent to or in contact with any surface thereof in a monopolar mode of operation.

In some embodiments, the lower jaw 350 can be operatively coupled to a control mechanism 354 such that actuation of the control mechanism can cause the lower jaw 350 to translate. The control mechanism 354 can have a variety of configurations. For example, the control mechanism 354 can be a mechanical switch that is configured to slide between a first position (FIG. 7A), which is associated with the extended configuration of the lower jaw 350, and a second position (FIG. 7B), which is associated with the retracted configuration of the lower jaw 350. As shown, the sliding direction of the mechanical switch in either case is indicated by arrow 355.

While energy can be delivered to tissue grasped between the upper and lower jaws 348, 350 via the upper law 348 when the device is in a monopolar energy delivery mode, the device 100 can be further configured with a bipolar energy delivery mode. For example, the lower jaw 350 can function as a return electrode. In such instances, when the lower jaw 350 is in the retracted configuration, the return electrode is electrically isolated from the upper jaw 348, and when the lower jaw 350 is in the extended configuration energy can be applied to tissue from the upper jaw 348 and can have a return path through the lower jaw 350.

Alternatively, the lower jaw 350 can be replaced with an ultrasonic blade 456, as shown in FIG. 9. The ultrasonic blade 456 can be configured to receive ultrasonic energy from a generator that is operably coupled an ultrasonic transducer disposed within the housing of the device. The ultrasonic transducer converts the electrical energy to ultrasonic vibrations that travel along the ultrasonic blade 456 so that the ultrasonic blade 456 can cut and/or coagulate tissue at the treatment site. The ultrasonic blade 456 can have a variety of shapes and sizes. For example, as shown, the ultrasonic blade 456 is curved. In certain embodiments, the ultrasonic blade 456 can be configured to retract relative to the instrument shaft 412 and relative to the upper jaw 448.

In one exemplary embodiment, a surgical device can include a switch mechanism configured to switch the device between a monopolar mode and a bipolar mode. In the monopolar mode, energy can be applied via a conductive member, in response to actuation of a sealing actuator, like sealing actuator 136 in FIGS. 1 and 3, and the return path is via a ground pad attached to the patient's body, usually at a site remote from the surgical site. In the bipolar mode, energy can be applied between the conductive member and a selectively conductive surface of a clamping element. Energy application may thus be achieved via the same actuation mechanism (the sealing actuator) regardless of whether the device is utilized in the bipolar or monopolar energy delivery mode. Such a configuration may help reduce user error and confusion during the high-stress experience of performing a surgical procedure.

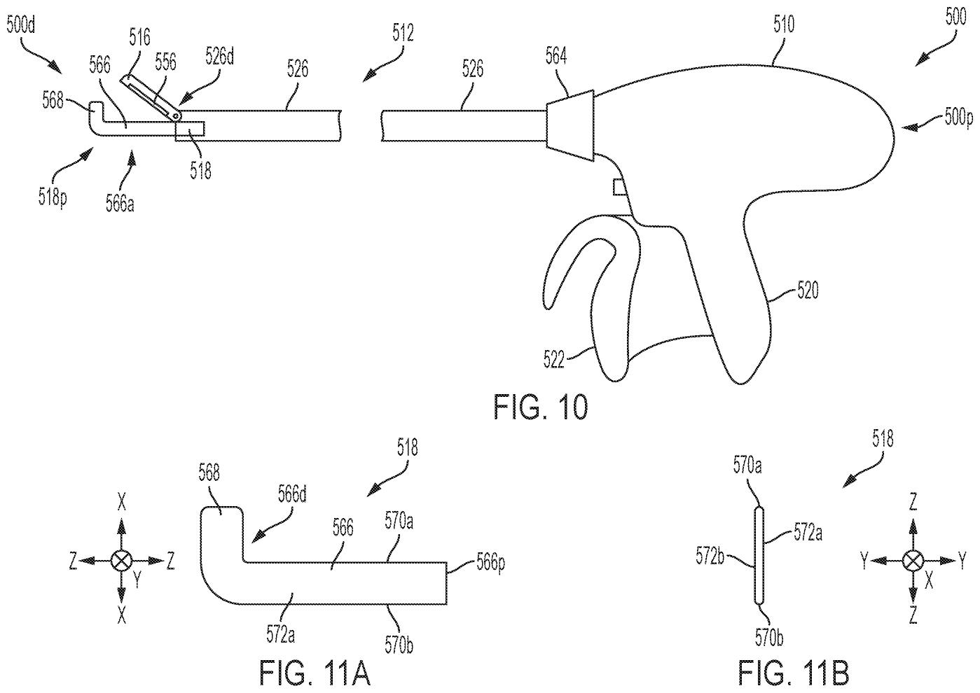

FIG. 10 illustrates an embodiment of a surgical device 500 capable of switching between monopolar and bipolar modes via a switching mechanism that can be activated through selective rotation of an outer sleeve 526 and a clamping element 516 about a stationary conductive member 518 that extends through at least a portion of outer sleeve 526 with a distal portion 518a extending distally from the outer sleeve 526. Aside from the differences described in detail below, the surgical device 500 can be similar to the surgical device 100 shown in FIG. 3 and therefore the similarities are not described in detail herein. Further, for purposes of simplicity, certain components of the surgical device 500 are not illustrated in FIG. 10.

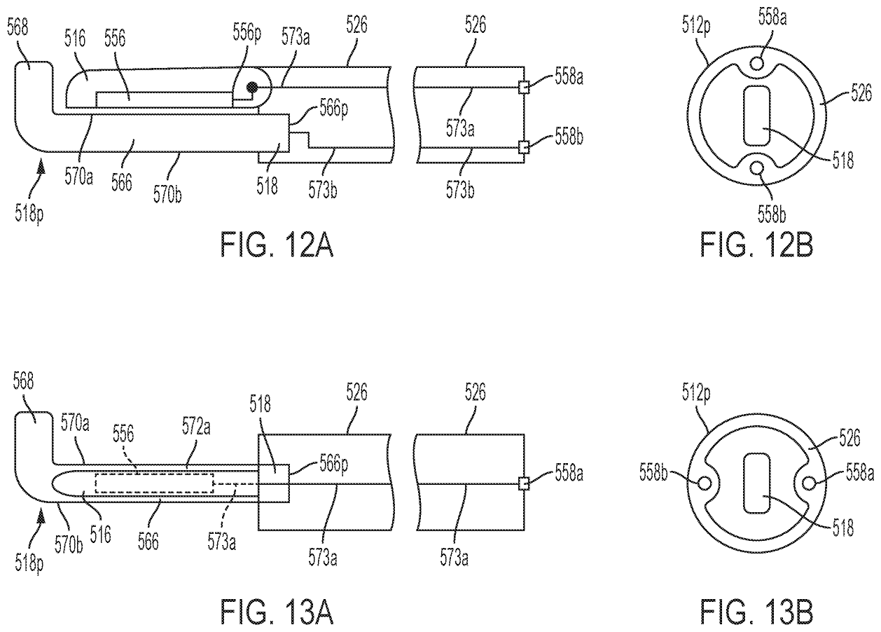

The clamping element 516 can be pivotally coupled to a distal portion 526d of the outer sleeve 526, and configured to be movable between open and closed positions. As shown in FIG. 10, the clamping element 516 is in an exemplary open position relative to the conductive member 518, and as shown in FIG. 12A, the clamping element 516 is shown in an exemplary closed position relative to the conductive member 518. Since the clamping element 516 is configured to rotate about the conductive member 518, the clamping element 516 can have open and closed positions relative to the conductive member 518, such as the exemplary closed positions illustrated in FIGS. 13A, 14A, and 15A.

While the clamping element 516 can have a variety of the configurations, the illustrated clamping element 516 is in the form of a selectively rotatable jaw that includes an electrode 556 that is configured to be selectively energized, as will be discussed in detail below. The clamping element 516 is operably coupled to a closure grip handle 522 that is coupled to a housing of 510, like housing 110 in FIG. 3. The closure grip handle 522 is configured to pivot relative to a stationary grip handle 520, like stationary grip handle 520 shown in FIG. 3. In an open position in which the closure grip handle 522 is angularly offset and spaced apart from the stationary grip handle 520, as shown in FIG. 10, causes the clamping element 516 to be in an open position, and thus spaced apart from the conductive member 518. When the closure grip handle 522 is in a closed position, and thus positioned adjacent to, or substantially in contact with, the stationary grip handle 520, causing the clamping element 516 to substantially close upon a portion of the distal portion of the conductive member 518.

Further, the clamping element 516 and outer sleeve 526 can be operably connected to a rotation knob 564 that is coupled to the housing 510. As such, the rotation of the rotation knob 564 can cause concurrent rotation of the clamping element 516 and outer sleeve 526 about the conductive member 518. In this way, rotation of the clamping element 516 and outer sleeve 526 about the conductive member 518 can be controlled by rotation of the rotation knob 564. While the rotation knob 564, and thus the clamping element 516 and outer sleeve 526, can rotate 360 degrees, in this illustrated embodiment, the rotation knob 564 is configured to rotate in about 90 degree increments such that the clamping element 516 can face, and thus close upon, four different surfaces of the conductive member 518, as shown in FIGS. 12A, 13A, 14A, and 15A. Alternatively, a trigger member (or other actuator) can be coupled to the housing 510 and be configured to translate or rotate through a range of depressed positions or pivoting positions, respectively, in which each position can be associated with a predetermined rotation position of the clamping element 516 relative to the conductive member 518.

The conductive member 518 can have a variety of configurations. In this illustrated embodiment, the conductive member 518 serves as an electrode having an L-shape with an elongate rod 566 and a hook or bent tip 568 on a distal end 566d thereof that extends at an approximately right angle thereto, as shown in FIGS. 10, 11A, 12A, 13A, 14A, and 15A. In some embodiments, at least a portion of the conductive member 518 can be coated with a protective, insulating material, e.g., Telefon, such that energy traveling through the conductive member 518 can be substantially directed to an exposed (uncoated), electrically-active portion of the conductive member 518, e.g., along an edge of the hook or bent tip 568. This can thus help protect various components within the device 500 and any secondary tissue from inadvertent electrical exposure while creating an easily-identifiable active distal end on the conductive member 518 for treatment of any target tissue. A person skilled in the art will appreciate that during use, the protective material can begin to burn off, e.g., along the coated edges of the conductive member 518, and therefore can result in other exposed, electrically-active portions of the conductive member 518. The conductive member 518 can be made from a variety of electrically-conductive materials, such as metal.

The elongate rod 566 can extend distally through a portion of the outer sleeve 526 such that a distal portion 566a of the elongate rod 566, and thus the conductive member 518, can extend distally outward from the outer sleeve 526. As a result, the hook or bent tip 568 can be positioned at the distal-most end of the surgical device 500, as shown in FIG. 10. While the elongate rod can have a variety of shapes and sizes, the elongate rod 566, as shown in detail in FIGS. 11A and 11B, has a substantially rectangular cross section. As such, the elongate rod 566 includes a first pair of opposing surfaces 570a, 570b and a second pair of opposing surfaces 572a, 572b that can each be used as an interface for treating tissue adjacent to or in direct contact therewith. In this illustrated embodiment, the first pair of opposing surfaces 570a, 570b are spaced apart from one another in the x-direction and the second pair of opposing surface 572a, 572b are spaced apart from one another along the y-direction. Further, the first part of opposing surfaces 570a, 570b have a width, e.g., in the y-direction, and the second pair of opposing surfaces 572a, 572b have a width, e.g., in the x-direction. As shown, the width of the first pair of opposing surfaces 570a, 570b is less than the width of the second pair of opposing surfaces 572a, 572b. In other embodiments, the width of the first pair of opposing surfaces 570a, 570b can be greater than the width of the second pair of opposing surfaces 572a, 572b.

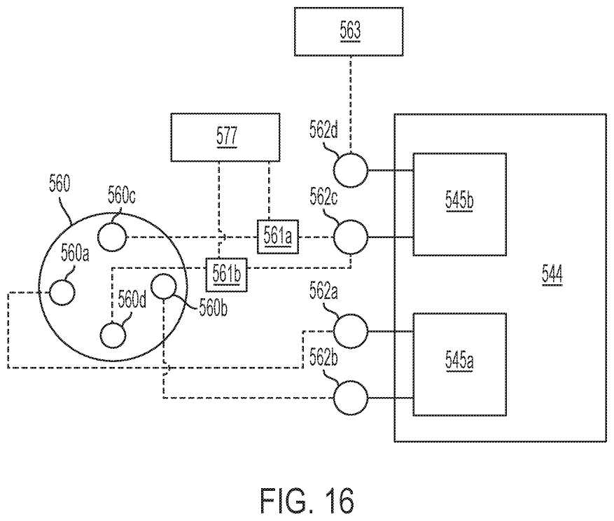

As shown in FIGS. 12A, 13A, 14A, and 15A, a proximal end 556p of the electrode 556 is coupled to a conductive element 573a, e.g., an electrical lead, wire, etc., that extends therefrom and is in electrical communication with a first shaft electrical contact 558a located at a proximal end 512p of the instrument shaft 512. Depending on the rotational position of the clamping element 516, the first shaft electrical contact 558a can be in electrical communication with either a first shroud electrical contact 560a or a second shroud electrical contact 560b that are each disposed within a housing 510, like housing 110 in FIG. 3, and associated with the bipolar mode of the surgical device 500. As shown in FIG. 16, the first and second shroud electrical contacts 560a, 560b, which are positioned at a distal end of a shroud 560 within the housing 510, are in electrical communication with corresponding first and second electrical terminals 562a, 562b of a generator 544. The first and second electrical terminals 562a, 562b are associated with a bipolar generator connection 545a.

Further, a proximal end 566p of the elongate rod 566 is coupled to a conductive element 573b, e.g., an electrical lead, wire, etc., that extends therefrom and is in electrical communication with a second shaft electrical contact 558b located at the proximal end 512p of the instrument shaft 512. As a result, the conductive member 518 is in electrical communication with the second shaft electrical contact 558b. Depending on the rotational position of the clamping element 516, the second shaft electrical contact 558b can be in electrical communication with the first shroud electrical contact 560a or the second shroud electrical contact 560b, or alternatively, a third shroud electrical contact 560c, or a fourth shroud electrical contact 560d that are disposed within the housing 510 and associated with the monopolar mode of the surgical device. As shown in FIG. 16, the third and fourth shroud electrical contacts 560c, 560d, which are positioned at a distal end of the shroud 560, can be in electrical communication with a corresponding third electrical terminal 562c of the generator 544. The third electrical terminal 562c is associated with the monopolar generator connection 545b. As further shown, the generator 544 includes a fourth electrical terminal 562d that is engaged with a ground 563, e.g., a patient pad that is placed on a patient's body prior to energizing the conductive member 518, and associated with the monopolar generator connection 545b.

As shown in FIGS. 12A, 13A, 14A, and 15A, for each rotational position of the clamping element 516 relative to the conductive member 518, the clamping element 516 can close upon one of the four surfaces 570a, 570b, 572a, 572b of the conductive member 518. Further, as shown in FIGS. 12B, 13B, 14B, and 15B, rotation of the clamping element between its four rotational positions also causes the first and second shaft electrical contacts 558a, 558b to rotate. As a result, since the electrical contacts 560a, 560b, 560c, and 560d within the housing 510 do not rotate, for each rotational position of the clamping element 516, the shaft electrical contacts 558a, 558b shift between the shroud electrical contacts 560a, 560b, 560c, 560d. This allows the surgical device 500 to switch between the monopolar mode and the bipolar mode.

The rotational position of the clamping element 516 with respect to the conductive member 518 and switching between the monopolar and bipolar modes of the surgical device 500 are discussed below. For the purposes of the following discussion, it is assumed that the operation of the surgical device 500 begins with the clamping element 516 positioned at a zero rotation position as illustrated in FIG. 12A, and that the clamping element 516 is rotated from its zero rotation position to non-zero rotation positions (e.g., a 90 degree rotation position, a 180 degree rotation position, and a 270 degree rotation position) in a counterclockwise direction when viewing the device 500 from its proximal end 500p. However, a person skilled in the art will appreciate that the zero rotation position can be defined at any predetermined rotation angle of the clamping element 516 with respect to the conductive member 518 and that the clamping element 516 can also be rotated from its zero rotation position to its non-zero rotation positions in a clockwise direction when viewing the device 500 from its proximal end 500p, which is opposite its distal end 500d. Further, a person skilled in the art will appreciate that the clamping element 516 can be rotated counterclockwise or clockwise to rotate the clamping element 516 between rotational positions.

In use, when the clamping element 516 is in the zero rotation position it faces, and therefore can close upon, the surface 570a of the elongate rod 566, as shown in FIG. 12A. As a result, the conductive member 518 can treat tissue adjacent to or in direct contact with the surface 570b of the elongate rod 566 and the hook tip 568. Further, the clamping element 516 can apply pressure to the conductive member 518 as the conductive member 518 applies energy to tissue. This can help enhance the sealing capabilities of the conductive member 518 during use.

Further, as shown in FIG. 12B, the first shaft electrical contact 558a, which is in electrical communication with the electrode 556 of the clamping element 516, is positioned at zero degrees, and the second shaft electrical contact 558b, which is in electrical communication with the conductive member 518, is positioned at 180 degrees. As a result, the electrode 556 and the conductive member 518 are in contact with the third shroud electrical contact 560c and the fourth shroud electrical contact 560d, respectively, such that the surgical device 500 is in a monopolar mode. This allows energy from the generator 544 to flow from the third electrical terminal 562c to only the conductive member 518, thereby energizing the conductive member 118, and then from the conductive member 118 into the tissue. The energy then flows from the tissue to the ground 563, e.g., through the patient to a ground pad positioned on the patient's body, and then returns to the generator 544 through the fourth electrical terminal 562d to complete the circuit. In the monopolar mode, the electrode 556 is de-energized, e.g., disconnected from the generator 544.

The electrode 556 can be disconnected from the generator using a variety of mechanisms. For example, a sensor, e.g., a rotary encoder, can be incorporated within the housing 510 and in communication with a local processor 577, or alternatively a remote processor. The sensor is configured to detect the rotation position of the clamping element 516, and thus the electrode 556, relative to the third and fourth electrical contacts 560c, 560d and transmit this data to the local processor 575. Further, as shown in FIG. 16, first and second switches 561a, 561b can be incorporated within the housing 510 and in communication with the local processor 575. Depending on the rotation position of the clamping element 516, the local processor can cause the first switch 561a or the second switch 561b to transition from a conductive state to a non-conductive state. In this illustrated embodiment, the electrical communication between the third shroud electrical contact 560c and the third electrical terminal 562c is dependent upon the state of the first switch 561a and the electrical communication between the fourth shroud electrical contact 560d and the fourth electrical terminal 562d is dependent upon the state of the second switch 561b. Further, the default state of the first and second switches 561a, 561b is their respective conductive states. However, a person skilled in the art will appreciate that in other embodiments, the default state of the first and second switches 561a, 561b can be their respective non-conductive states.

In use, when the clamping element 516 is determined to be in the zero rotation position (FIG. 12A), the local processor 575 can prompt the first switch 561a to transition from its conductive state to its non-conductive state while the second switch 561b remains in its conductive state. This terminates electrical communication between the third shroud electrical contact 560c, which is ultimately in contact with electrode 556 when the clamping element 516 is in this zero rotation position, and the third terminal contact 562c. As a result, the electrode 556 is disconnected from the generator 544 and energy can no longer flow from the third electrical terminal 562c to the electrode 556.

When the clamping element 516 is rotated to a first rotation position, which as illustrated in FIG. 13A, is a 90 degree counterclockwise rotation position, it faces, and therefore can close upon, the surface 572a of the elongate rod 566 to grasp and treat tissue therebetween in the bipolar mode. This is because, as shown in FIG. 13B, the first shaft electrical contact 558a, which is in electrical communication with the electrode 556 of the clamping element 516, is positioned at 90 degrees, and the second shaft electrical contact 558b, which is in electrical communication with the conductive member 518, is positioned at 270 degrees. As a result, the electrode 556 and the conductive member 518 are in contact with the first shroud electrical contact 560a and the second shroud electrical contact 560b, respectively, such that the surgical device 500 is in a bipolar mode. This allows energy from the generator 544 to flow from the second electrical terminal 562b to the conductive member 518, through tissue that is grasped between the conductive member 518 and the clamping element 516 to the electrode 556. The energy than flows from the electrode 556 to the first electrical terminal 562a to complete the circuit. As such, the electrode 556 of the clamping element 516 functions as the return electrode for the bipolar circuit. Alternatively, the conductive member 518 can function as the return electrode for the circuit in which energy would flow in the reverse direction.

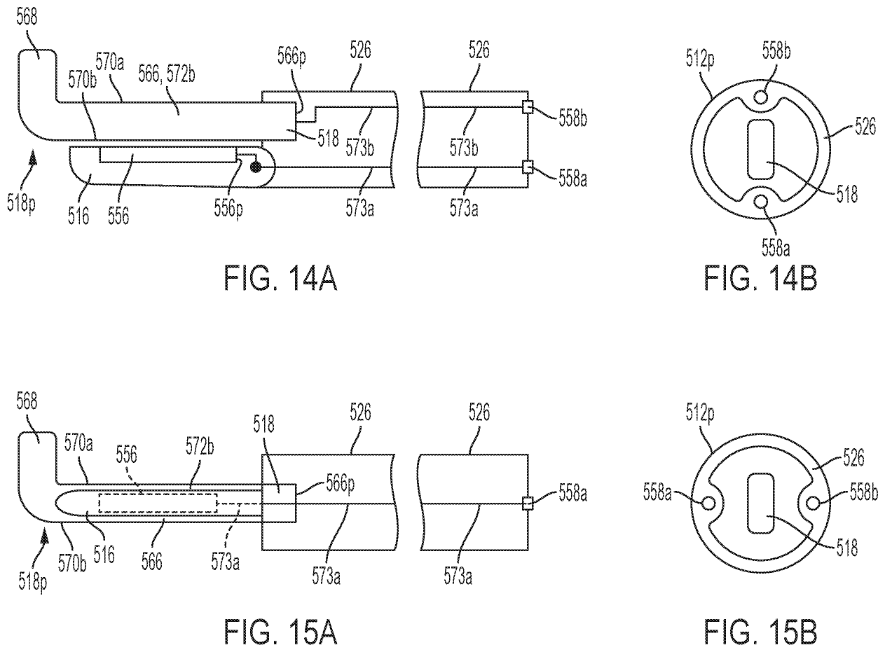

When the clamping element 516 is rotated to a second rotation position, which as illustrated in FIG. 14A, is a 180 degree counterclockwise rotation position, it faces, and therefore can close upon, the surface 570b of the elongate rod 566. As a result, the conductive member can treat tissue adjacent to or in direct contact with the surface 570a of the elongate rod 566 and the hook tip 568. Further, the clamping element 516 can apply pressure to the conductive member 518 as the conductive member 518 applies energy to tissue. This can help enhance the sealing capabilities of the conductive member 518 during use.

Further, as shown in FIG. 14B, the first shaft electrical contact 558a, which is in electrical communication with the electrode 556 of the clamping element 516, is positioned at 180 degrees, and the second shaft electrical contact 558b, which is in electrical communication with the conductive member 518, is positioned at zero degrees. As a result, the electrode 556 and the conductive member 518 are in contact with the fourth shroud electrical contact 560d and the third shroud electrical contact 560c, respectively, such that the surgical device 500 is switched back into the monopolar mode. And, as discussed above, when in the monopolar mode, only the conductive member 518 is energized to deliver energy to, and thus treat, tissue adjacent to or in direct contact with the conductive member 518. In this illustrated embodiment, when the clamping element 516 is determined to be in the 180 degree rotation position (FIG. 14A), the local processor 575 can cause the second switch 561b to transition from its conductive state to its non-conductive state while the first switch 561a remains in its conductive state. This terminates electrical communication between the fourth shroud electrical contact 560d, which is ultimately in contact with electrode 556 when the clamping element 516 is in this 180 degree rotation position, and the third terminal contact 562c. Thus, when the clamping element 516 is in either the zero rotation position or the second rotation position, the surgical device 500 is in the monopolar mode.

When the clamping element 516 is rotated to a third rotation position, which as illustrated in FIG. 14A, is a 270 degree counterclockwise rotation position, it faces, and therefore can close upon, the surface 572b of the elongate rod 566 to grasp and treat tissue therebetween in the bipolar mode. As such, the surgical device is switched back into the bipolar mode. This is because, as shown in FIG. 14B, the first shaft electrical contact 558a, which is in electrical communication with the electrode 556 of the clamping element 516, is positioned at 270 degrees, and the second shaft electrical contact 558b, which is in electrical communication with the conductive member 518, is positioned at 90 degrees. Thus, when the clamping element 516 is in either the first or third rotation position, the surgical device 500 is in the bipolar mode. And, as discussed above, when in the bipolar mode, energy can be applied between the electrode 556 and the conductive member 518, and thus treat tissue grasped therebetween.

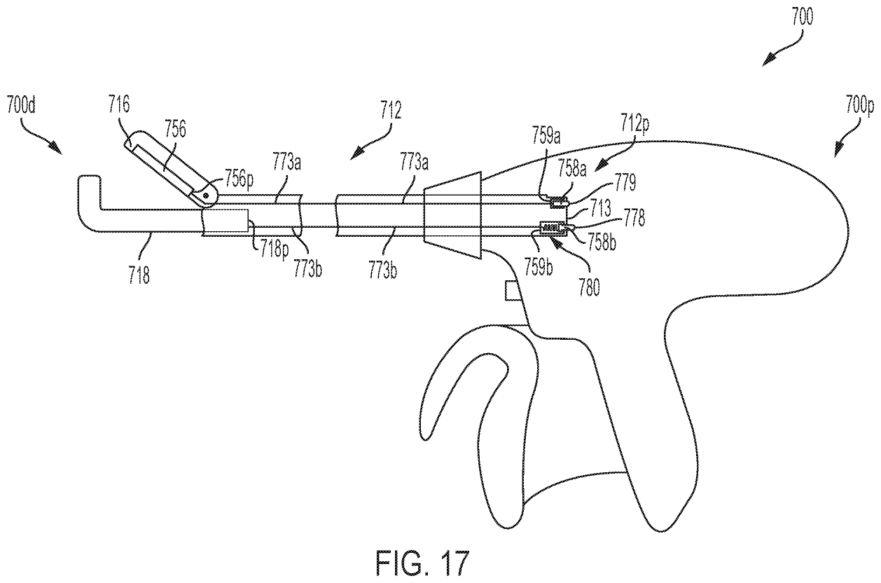

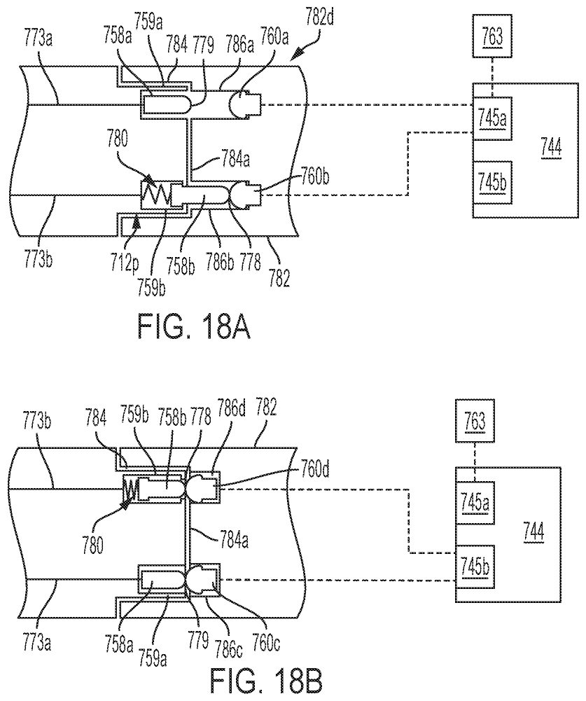

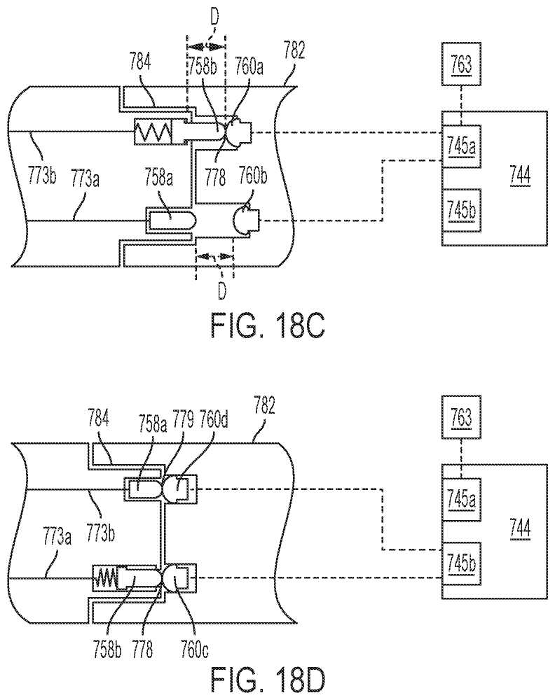

FIG. 17 illustrates an embodiment of a surgical device 700 having shaft electrical contacts 758a, 758b that are configured for engagement to shroud electrical contacts, like shroud electrical contacts 760a, 760b, 760c, 760d shown in FIGS. 18A-18D, in a different manner as compared to shaft electrical contacts 558a, 558b and shroud electrical contacts 560a, 560b, 560c, 560d of surgical device 500 discussed above. Aside from the differences described in detail below, the surgical device 700 can be similar to the surgical device 500 shown in FIG. 10 and therefore the similarities are not described in detail herein. Further, for purposes of simplicity, certain components of the surgical device 700 are not illustrated in FIG. 17.

As shown in FIG. 17, a proximal end 756p of the electrode 756 is coupled to a first conductive element 773a, e.g., an electrical lead, wire, etc., that extends therefrom and is in electrical communication with a first shaft electrical contact 758a located within a first channel 759a extending from a proximal-most end 713 of the instrument shaft 712. Further, a proximal end 718p of the conductive member 718 is coupled to a second conductive element 773b, e.g., an electrical lead, wire, etc., that extends therefrom and is in electrical communication with a second shaft electrical contact 758b located within a second channel 759b extending from the proximal end 712p of the instrument shaft 712. As shown, the second shaft electrical contact 758b is biased in an extended position (e.g., towards the proximal end 700p of the device 700) such that a terminal contacting surface 778 of the second shaft electrical contact 758b can extend past a terminal contacting surface 779 of the first shaft electrical contact 758a. In particular, the proximal end 780 of the second conductive element 773b is the form of a spring.

The proximal end 712p of the instrument shaft 712 is configured to couple to a distal end 782d of a shroud 782 that is positioned within the housing 510. While the shroud 782 can have a variety of configurations, in this illustrated embodiment, the shroud 782 is in the form of a hollow elongated tube. As shown in FIGS. 18A-18D, a notch 784 is defined within the distal end 782d of the shroud 782 and configured to receive the proximal end 712p of the instrument shaft 712. In this manner, the proximal end 712p of the instrument shaft 712 is radially confined within the notch 784. Further, as shown, the shroud 782 includes first and second shroud electrical contacts 760a, 760b that are in electrical communication with a monopolar generator connection 745a of a generator 744, and third and fourth shroud electrical contacts 760c, 760d that are in electrical communication with a bipolar generator connection 745b of the generator 744. As such, the first and second shroud electrical contacts 760a, 760b are associated with a monopolar mode of the device 700, and the third and fourth shroud electrical contacts 760c, 760d are associated with a bipolar mode of the device 700.

FIGS. 18A-18D illustrate the different positions of the shaft electrical contacts 758a, 758b relative to the shroud electrical contacts 760a, 760b, 760c, 760d when the clamping element 716 is in different rotation positions. For the purposes of the following discussion, it is assumed that the operation of the surgical device 700 begins with the clamping element 716 positioned at a zero rotation position, as illustrated in FIGS. 17 and 18A, and that the clamping element 716 is rotated from its zero rotation position to non-zero rotation positions (e.g., a 90 degree rotation position, a 180 degree rotation position, and a 270 degree rotation position) in a counterclockwise direction when viewing the device 700 from its proximal end 700p. While not shown in FIGS. 18B-18D, the clamping element 716 is in is in a 90 degree rotation position (first rotation position) in FIG. 18B, which is similar to the rotation position of clamping element 516 in FIG. 12B, in a 180 degree rotation position in FIG. 18C, which is similar to the rotation position of clamping element 516 in FIG. 12C, and in a 270 degree rotation position in FIG. 18D, which is similar to the rotation position of clamping element 516 in FIG. 12D. However, a person skilled in the art will appreciate that the zero rotation position can be defined at any predetermined rotation angle of the clamping element 716 with respect to the conductive member 718 and that the clamping element 716 can also be rotated from its zero rotation position to its non-zero rotation positions in a clockwise direction when viewing the device 500 from its proximal end 700p, which is opposite its distal end 700d. Further, a person skilled in the art will appreciate that the clamping element 716 can be rotated counterclockwise or clockwise to rotate the clamping element 716 between rotational positions.

As shown in FIGS. 18A and 18C, the first and second shroud electrical contacts 760a, 760b are positioned within respective recesses 786a, 786b formed within the shroud 782. The first and second shroud electrical contacts 760a, 760b are each spaced at a distance D1, D2 from the base 784a of the notch 784. As a result, the first shaft electrical contact 758a is unable to make contact with either the first shroud electrical contacts 760a or the second shroud electrical contact 760b, and is therefore disconnected from the generator 744, when the clamping element 716 is in the zero rotation position (FIG. 18A) and in the 180 degree rotation position (FIG. 18C). In this illustrated embodiment, distance D1 and distance D2 are equal, whereas in other embodiments, distance D1 can be shorter or longer than distance D2.

Further, when the clamping element 716 is in the zero rotation position (FIG. 18A), the terminal end 778 of the second shaft electrical contact 758b is in contact with the second shroud electrical contact 760b, and when the clamping element 716 is rotated to the 180 degree rotation position (FIG. 18C), the terminal end 778 of the second shaft electrical contact 758b is in contact with the first shroud electrical contact 760a. This allows energy from the generator 744 to flow from the monopolar generator connection 745a to only the conductive member 718, thereby energizing the conductive member 718, and then from the conductive member 718 into the tissue. The energy then flows from the tissue to the ground 763, e.g., through the patient to a ground pad positioned on the patient's body, and then returns to the generator 744 to complete the circuit. As such, when the clamping element 716 is in the zero rotation position (FIG. 18A) or the 180 degree rotation position (FIG. 18C), the device 700 is in the monopolar mode.

As shown in FIGS. 18B and 18D, the third and fourth shroud electrical contacts 760c, 760d are positioned within respective recesses 786c, 786d formed within the shroud 782. The terminal ends 778, 779, of the first and second shroud electrical contacts 760a, 760b each extend into the notch 784. In this way, when the clamping element 716 is rotated into the 90 degree position (FIG. 18B), the first shaft electrical contact 758a is in contact with the third shroud electrical contact 760c and the second shaft electrical contact 758b is in a retracted position and in contact with the fourth shroud electrical contact 760d. Further when the clamping element 716 is rotated into the 270 degree position (FIG. 18D), the first shaft electrical contact 758a is in contact with the third shroud electrical contact 760c and the second shaft electrical contact 758b is a retracted position and in contact with the fourth shroud electrical contact 760d. This allows energy from the generator 744 to flow from the bipolar generator connection 745b to the conductive member 718, through tissue that is grasped between the conductive member 718 and the clamping element 716, and to the electrode 756. The energy than flows from the electrode 756 back to bipolar generator connection 745b to complete the circuit. As such, the electrode 756 of the clamping element 716 functions as the return electrode for the bipolar circuit. Alternatively, the conductive member 718 can function as the return electrode for the circuit in which energy would flow in the reverse direction. Thus, when the clamping element 716 is in either the 90 degree or 270 degree rotation position, the surgical device 700 is in the bipolar mode.

The devices disclosed herein can be designed to be disposed of after a single use, or they can be designed to be used multiple times. In either case, however, the device can be reconditioned for reuse after at least one use. Reconditioning can include any combination of the steps of disassembly of the instrument, followed by cleaning or replacement of particular pieces and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of an device can utilize a variety of techniques for disassembly, cleaning/replacement, and reassembly. Use of such techniques, and the resulting reconditioned device, are all within the scope of the present application.

It will be appreciated that the terms “proximal” and “distal” are used herein with reference to a user, such as a clinician, gripping a handle of an instrument. Other spatial terms such as “front” and “rear” similarly correspond respectively to distal and proximal. It will be further appreciated that for convenience and clarity, spatial terms such as “vertical” and “horizontal” are used herein with respect to the drawings. However, surgical instruments are used in many orientations and positions, and these spatial terms are not intended to be limiting and absolute.

For purposes of describing and defining the present teachings, it is noted that unless indicated otherwise, the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety. Any patent, publication, or information, in whole or in part, that is said to be incorporated by reference herein is incorporated herein only to the extent that the incorporated material does not conflict with existing definitions, statements, or other disclosure material set forth in this document. As such the disclosure as explicitly set forth herein supersedes any conflicting material incorporated herein by reference.

What is claimed is:

1. A surgical device, comprising:
a housing having an instrument shaft extending therefrom, the instrument shaft including an inner sleeve and an outer sleeve that is partially disposed around the inner sleeve, and
an end effector assembly having a clamp arm extending distally from the outer sleeve and is movable between open and closed positions, and a conductive member extending distally from the inner sleeve,
wherein the clamp arm has a tissue contacting surface and is configured to translate between a retracted configuration in which it overlaps with and is closed upon a distal portion of the inner sleeve and an extended configuration in which it at least partial overlaps with the conductive member and configured to open and close upon the conductive member.

2. The device of claim 1, wherein an upper portion of the clamp arm is pivotally connected to a distal portion of the outer sleeve and the outer sleeve is movable between retracted and extended configurations.

3. The device of claim 2, wherein a lower portion of the clamp arm includes a pin configured to travel within a cam slot formed within a portion of the inner sleeve, and wherein when the pin is at a proximal-most end of the cam slot, the clamp arm is retracted and closed upon the distal portion of the inner sleeve and wherein distal movement of the pin within the cam slot moves the clamp arm to an open position and advances the clamp arm distally towards the conductive member.

4. The device of claim 3, wherein further distal movement of the pin to a distal-most end of the cam slot moves the clamp arm distally into alignment with the conductive member and causes the clamp arm to close upon the conductive member.

5. The device of claim 1, wherein the conductive member is a monopolar cutting blade.

6. The device of claim 1, wherein when the clamp arm is in the retracted configuration the device is configured to treat tissue in a monopolar energy delivery mode with the conductive member.

7. The device of claim 1, wherein when the clamp arm is in the extended configuration the device is configured to treat tissue disposed between the clamp arm and the conductive member.

8. The device of claim 1, wherein the tissue contacting surface of the clamp arm is conductive, and wherein when the clamp arm is in the extended configuration the device is configured to treat tissue disposed between the clamp arm and the conductive member in a bipolar energy delivery mode.

9. The device of claim 1, wherein the end effector assembly further includes a support structure extending distally from the inner sleeve and positioned below and in contact with the conductive member.

10. The device of claim 1, wherein the end effector assembly further includes at least one slot that extends through at least a portion of at least one of the clamp arm and the conductive member, wherein the slot is configured to receive a cutting element.

11. A surgical device, comprising:
an instrument shaft operably coupled to and extending from a housing, the instrument shaft including an outer sleeve and a clamp arm pivotably coupled to a distal end of the outer sleeve, the clamp arm having a selectively conductive surface formed at least partially thereon; and
a conductive member extending through the outer sleeve;
wherein the clamp arm and outer sleeve are configured to selectively rotate about the conductive member to cause the device to move between configurations for a monopolar mode of operation and a bipolar mode of operation.

12. The device of claim 11, wherein when in the monopolar mode, the clamp arm is de-energized and the conductive member is configured to apply energy to tissue disposed between the clamp arm and the conductive member, and wherein when in the bipolar mode, energy is delivered between the clamp arm and the conductive member to tissue disposed therebetween.

13. The device of claim 11, wherein when in the monopolar mode, the clamp arm is de-energized and the selectively conductive surface of the clamp arm faces a first surface of the conductive member, and wherein when in the bipolar mode, the clamp arm is energized and the selectively conductive surface faces a second surface of the conductive member.

14. The device of claim 12, wherein the first surface has a width that is less than the second surface.

15. The device of claim 11, wherein when in the monopolar mode, the clamp arm is de-energized and configured to apply pressure to tissue disposed between the clamp arm and the conductive member.

16. The device of claim 11, wherein the conductive member is substantially L-shaped.

17. A surgical method, comprising:
positioning at least one of a clamp arm and a conductive member of an end effector assembly of a surgical device in contact with tissue, the clamp arm being coupled to a distal portion of an outer sleeve of the surgical device and the conductive member extending distally from an inner sleeve of the surgical device;
actuating an energy source to supply energy to at least one of the clamp arm and the conductive member to treat tissue located adjacent to or in direct contact therewith; and
longitudinally translating the clamp arm or the conductive member from a retracted configuration to an extended configuration to position tissue between the clamp arm and the conducive member.

18. The method of claim 17, wherein the clamp arm is longitudinally translated from its retracted configuration to its extended configuration, and the method further comprises moving the clamp arm from an open position to a closed position to grasp tissue positioned between the clamp arm and the conducive member.

19. The method of claim 17, wherein when the surgical device is in a monopolar energy delivery mode, the step of actuating the energy source comprises supplying energy only to the conductive member to treat tissue located adjacent to or in direct contact therewith.

20. The method of claim 17, wherein the clamp arm includes an electrode and when the surgical device is in a bipolar energy deliver mode, the method further comprises actuating the energy source to supply energy to the electrode or the conductive member to treat tissue grasped therebetween.

21. The method of claim 17, wherein when the surgical device is in a monopolar energy mode, the step of actuating the energy source comprises supplying energy only to an electrode of the clamp arm.

22. The method of claim 17, further comprising longitudinally translating a cutting element from a retracted position to an extended position to cut tissue positioned between the clamp arm and the conductive member.
